(19) United States
(12) Patent Application Publication
(10) Pub. No.: US 2006/0024253 A1
(43) Pub. Date: Feb. 2, 2006

(54) Use of a photocatalyst for the treatment of oily hair

(75) Inventors: Geraldine Fack, Levallois (FR); Henri Samain, Bievres (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/526,203

(22) PCT Filed: Jan. 26, 2004
(86) PCT No.: PCT/FR04/00172

§ 371 Date: Mar. 16, 2005

(51) Int. Cl. A61K 8/29

(52) U.S. Cl. 424/70.1

(57) ABSTRACT

The present invention relates to the use of a photocatalyst in hair compositions intended to decrease and/or to eliminate the unwanted organic compounds on the hair, in particular sebum. The invention relates in particular to the use of a photocatalyst in hair compositions intended to decrease and/or delay regressing of hair.

The present invention relates to the use of a photocatalyst in hair compositions intended to decrease and/or to eliminate the unwanted organic compounds on the hair, in particular sebum. The invention relates in particular to the use of a photocatalyst in hair compositions intended to decrease and/or delay regreasing of the hair, and to a method using this compound.

Hair has a tendency to lose some of its qualities under the action of factors such as natural regressing, sweat, the elimination of squamae, pollution, humidity, and the like. These factors are harmful to the visual appearance and to the feel of the hair. Thus, regressing (and possibly pollution) weigh down the hair, which has a tendency to clump together. The hair is then difficult to style, it has an unpleasant greasy sheen and is waxy to the touch, which is also unpleasant.

The extent of the consequences of these factors, which are almost all inevitable, is very variable. It depends, for example, on the quality of the hair, on its length, and on the hairstyle adopted.

Be that as it may, shampoos are used to combat these inconveniences. In fact, washing with detergent compositions is very effective; it makes it possible to remove dirt and dandruff and to relax the hair. It is then possible, during drying, to return the hair to the desired shape. However, the beneficial effect of the shampoo fades and, in a few days, the problems described above return. Consequently, there is a tendency to increase the frequency of shampooing.

To shampoo the hair, it is necessary to have a source of water, preferably hot or warm water.

Shampoo compositions are based on large amounts of surfactants which can generate inconveniences such as stinging of the scalp or of the eyes.

The use of “dry” shampoos has already been proposed in order to clean the hair more rapidly and to avoid wetting the hair. This technique consists in spraying absorbent particles onto the hair and then in actively brushing the hair in order to remove the particles. However, in general, it is very difficult to obtain complete removal of the particles. The results are not very satisfactory. The hair is not very shiny and it is rough to the touch.

Moreover, it is known that it is possible to remove all or some of the foreign or unwanted bodies from the hair (including sebum, pollution, etc.) by oxidation.

The technique consists in bringing an oxidant into contact with the compounds to be removed on the hair. This technique is not satisfactory since its effect is immediate and cannot be extended over time given the lack of persistence of the oxidant, associated with the complete consumption thereof during the reaction. Moreover, the oxidants used often have a harmful action on the hair.

The aim of the present invention is in particular to solve the above problems.

More precisely, the present invention aims to propose a method of treating, in particular cosmetically, human hair in order to decrease and/or delay regressing of the hair.

Now, subsequent to considerable research carried out on the subject, it has now been found by the applicant, entirely unexpectedly and surprisingly, that this aim, and others, can be achieved by using cosmetic compositions intended for treating the hair, comprising, in a cosmetically acceptable medium, in particular aqueous or aqueous-alcoholic medium, at least one specific photocatalyst. This discovery forms the basis of the present invention.

The hair treated according to the invention has the characteristics of clean hair: the hair is neutral to the touch (not coated), the hair is shiny (the greasy appearance has disappeared), the hair is airy and there is no dandruff. The regressing is delayed and the hair keeps its clean appearance for several days, which makes it possible to space out the shampooings.

They nevertheless have the advantage of conserving a natural and pleasant feel to the hair.

A subject of the present invention is the use of a photocatalyst in hair compositions intended to decrease and/or to eliminate the unwanted organic compounds on human hair, in particular sebum, said photocatalyst being chosen from:
1) water-insoluble compounds comprising at least one element of the following columns of the Periodic Table of Elements: IIIb, Vb, VIb, VIIb, VIII (3 columns), Ib and Va;
2) water-insoluble sulphides, carbides and phosphides comprising at least one element of the following columns of the Periodic Table of Elements: IIb, IIIa, IVa, IVb;
3) water-insoluble composites comprising at least one element chosen from:
titanium, zinc, silicon, aluminium, magnesium, sodium, calcium and zirconium.

A subject of the invention is in particular the use of a photocatalyst in hair compositions intended to decrease and/or delay regressing of human hair, said photocatalyst being chosen from:
1) water-insoluble compounds comprising at least one element of the following columns of the Periodic Table of Elements: IIb, IIIb, IVb, Vb, VIb, VIIb, VIII (3 columns), Ib and Va;
2) water-insoluble sulphides, carbides and phosphides comprising at least one element of the following columns of the Periodic Table of Elements: IIb, IIIa, IVa, IVb;
3) water-insoluble composites comprising at least one element chosen from:
titanium, zinc, silicon, aluminium, magnesium, sodium, calcium and zirconium.

A subject of the present invention is also a method of cosmetically treating human hair, intended to decrease and/or to eliminate the unwanted organic compounds on human hair, in particular sebum, said method being characterized in that it comprises the following steps:
i) a composition comprising a photocatalyst as described below, in a cosmetically acceptable medium, is applied to the hair, in particular dry hair,
ii) optionally, the hair is massaged using the fingers,
iii) optionally, the hair is dried.

A subject of the present invention is also a method of treating the hair in order to decrease and/or delay regreasing of human hair, said method being characterized in that it comprises the following steps:
i) a composition comprising a photocatalyst as described below, in a cosmetically acceptable medium, is applied to the hair, in particular dry hair,
ii) optionally, the hair is massaged using the fingers,
iii) optionally, the hair is dried.

A subject of the present invention is also cosmetic compositions comprising, in a cosmetically acceptable medium, at least one photocatalyst chosen from:
1) water-insoluble compounds comprising at least one element of the following columns of the Periodic Table of Elements: Vb, VIIb, VIII (2nd and 3rd columns starting from the left), and Va,
2) water-insoluble sulphides and phosphides comprising at least one element of the following columns of the Periodic Table of Elements: IIb, IIIa, IVa, IVb,
3) water-insoluble composites chosen from: TiO₂+ZnO, TiO₂+CuO, TiO₂+RuO₂, TiO₂+SnO₂, TiO₂+MoO₃, TiO₂+WO₃, TiO₂+GeO₂, TiO₂+MoO₃, TiO₂+WO₃, TiO₂+GeO₂, and TiO₂ surface-coated with compounds comprising at least one element chosen from Al, Zn, Zr, Cr, V, Nv, Fe, Cu, Co, Ni and Mn.

For the purposes of the present invention, the term “photocatalyst” is intended to mean a catalyst the activity of which consists in accelerating a reaction in the presence of electromagnetic radiation having a wavelength of between 200 and 700 nanometres.

The Periodic Table of Elements used is the CAS (Chemical Associated Society) version. It is in particular included in the manual “The Merck Index”, 11 ed., 1989.

The term “water-insoluble” is intended to mean any compound which, at a concentration greater than or equal to 0.1% by weight in water at 25° C., does not form, to the naked eye, a transparent isotropic solution.

In accordance with the use according to the invention, the photocatalysts are chosen from:
1) water-insoluble compounds comprising at least one element chosen from vanadium, niobium, tantalum, molybdenum, tungsten, manganese, iron, ruthenium, cobalt, nickel, copper, silver, gold, bismuth, titanium, zinc and zirconium;
2) water-insoluble sulphides, carbides and phosphides comprising at least one element of the following columns of the Periodic Table of Elements: IIb, IIIa, IVa, IVb;
3) water-insoluble composites comprising at least one element chosen from:
   titanium, zinc, silicon, aluminium, magnesium, sodium, calcium and zirconium.

Among the compounds of family 1), mention may in particular be made of the following compounds:
V₂O₅, CeO₂, Nb₂O₅,
WO₃, Na₄W₁₀O₃₂
MoO ₃, MOS₂, RuO₂, Mn₂O₃, Bi₂O₃,
Fe₂O₃, Co₃O₄,
CuInS₂, CuIn₅S₈
metals such as Ag, Cu, Au, Ni.

Among the compounds of family 2), mention may in particular be made of GaP, SiC, CdS and TiS.

For the purpose of the present invention, the term “composite” is intended to mean a macroscopic combination of two or more materials. The macroscopic characteristic excludes alloys.

Among the compounds of family 3), mention may in particular be made of the following composites: TiO₂+ZnO, TiO₂+CuO, TiO₂+RuO₂, TiO₂+SnO₂, TiO₂+MoO₃, TiO₂+WO₃, TiO₂+GeO₂, TiO₂+MoO₃, TiO₂+WO₃, TiO₂+GeO₂, and TiO₂ surface-coated with compounds comprising at least one element chosen from Al, Zn, Zr, Cr, V, Nv, Fe, Cu, Co, Ni and Mn.

The photocatalysts of family 1) which can be used in the compositions according to the invention are chosen from:
V₂O₅, Nb₂O₅,
RuO₂, Mn₂O₃, Bi₂O₃,
Co₃O₄,
CuInS₂, CuIn₅S₈.

The photocatalysts of family 2) which can be used in the compositions of the invention are chosen from:
GaP, CdS and TiS.

Among the compounds of family 3) which can be used in the compositions of the invention, mention may in particular be made of the following composites: TiO₂+ZnO, TiO₂+CuO, TiO₂+RuO₂, TiO₂+SnO₂, TiO₂+MoO₃, TiO₂+WO₃, TiO₂+GeO₂, TiO₂+MoO₃, TiO₂+WO₃, TiO₂+GeO₂, and TiO₂ surface-coated with compounds comprising at least one element chosen from V, Nv, Fe, Cu and Mn.

The photocatalyst is generally present in the composition in an amount of between 0.1 and 20% by weight relative to the total weight of the composition, and preferably of between 0.2 and 10% by weight, and more particularly of 0.2 to 3% by weight.

The cosmetically acceptable medium may consist only of water, or of a mixture of water and of one or more cosmetically acceptable solvents, or of one or more cosmetically acceptable solvents, such as a C₁-C₄ lower alcohol, for instance ethanol, isopropanol, tert-butanol or n-butanol; alkylene glycols such as propylene glycol, or glycol ethers. The water preferably represents from 30 to 98% by weight, and preferably from 50 to 98% by weight relative to the total weight of the composition.

The pH of the compositions used according to the invention is generally between 2 and 12.

The composition of the invention may also contain at least one additive chosen from thickeners, fragrances, pearlescent agents, preserving agents, sunscreens, anionic, nonionic, amphoteric or cationic surfactants, anionic, nonionic or amphoteric polymers, cationic polymers, proteins, protein hydrolysates, ceramides, pseudoceramides, C₁₆-C₄₀ linear or branched-chain fatty acids such as 18-methyl eicosanoic acid, hydroxy acids, vitamins, provitamins such as panthenol, silicones, plant oils, mineral oils and synthetic oils, anti-dandruff agents and any other additive conventionally used in the cosmetics field which does not affect the stability or the properties of the compositions according to the invention.

These additives are optionally present in the composition according to the invention in proportions which may range from 0.001 to 50% by weight relative to the total weight of the composition. The precise amount of each additive is readily determined by those skilled in the art according to its nature and its function.

The composition can be applied with the hands, with a spraying device, with an aerosol, with an applicator tip, with a dispensing comb or with a towel impregnated with the composition. This application may or may not be followed by rinsing.

In the following examples, given by way of non-limiting illustration, a concrete composition in accordance with the invention is given.

### EXAMPLE

A composition containing 5% by weight of silver nanoparticles (15 nm) in colloidal aqueous solution, sold by the company Advanced Nanoproducts under the reference “Ag Sol”, was used.

This solution was deposited in a proportion of 1 g on a clean lock of natural hair of 2.7 g, i.e. an amount of 18.5 mg of silver per g of hair. The lock was dried under a hood for 30 minutes at 60° C. A placebo lock was also effected using water. The locks were exposed to daylight for 24 hours. Fine droplets of artificial sebum were then sprayed onto the lock (T0).

The lock was then exposed for 6 hours (T6) to the Sun Test (Hanau) equipped with a Xenon lamp allowing UV rays to pass within a wavelength range of between 300 and 830 nm. The lamp was cooled during the test by means of a stream of air approximately 60 m³/hour. A second stream of air approximately 60 m³/hour cooled the locks. The light intensity on the locks was approximately 150 lux. The intensity of irradiation was approximately 830 W/m² (5% RH). A control lock was left in a dark glove box away from any source of radiation.

| | Product applied | Application | |
|---|---|---|---|
| | to the mesh | of sebum | Exposure of the locks |
| Lock 1 | Silver nanoparticles | yes | 6 hours of Sun Test |
| Lock 2 | Silver nanoparticles | yes | 6 hours darkness |
| Lock 3 | Placebo (water) | yes | 6 hours of Sun Test |
| Lock 4 | Placebo (water | yes | 6 hours darkness |
| Lock 5 | Nothing | yes | 6 hours of Sun Test |
| Lock 6 | Nothing | yes | 6 hours darkness |

The locks were examined by the assessors after 6 hours of exposure, followed by 15 minutes of conditioning at ambient temperature.

The sensory comparison of the locks was carried out according to a triangular test by individuals.

Lock 1 appeared to be significantly less greasy (to the touch and visually) and cleaner than locks 2 to 6.

1-14. (canceled)

15. A method for cosmetically treating human hair, said method comprising:
applying a hair composition to said human hair, wherein said hair composition comprises, in a cosmetically acceptable medium, at least one photocatalyst selected from the group consisting of:
1) one or more water-insoluble compounds comprising at least one element selected from columns IIIb, Vb, VIb, VIb, VIII (3 columns), Ib and Va of the Periodic Table of Elements;
2) one or more water-insoluble sulphides, carbides and phosphides comprising at least one element selected from columns IIb, IIIa, IVa, and IVb of the Periodic Table of Elements; and
3) one or more water-insoluble composites comprising at least one element selected from the group consisting of titanium, zinc, silicon, aluminium, magnesium, sodium, calcium and zirconium.

16. The method according to claim 15, further comprising massaging said human hair using one or more fingers.

17. The method according to claim 15, further comprising drying said human hair.

18. The method according to claim 15, wherein said method decreases and/or eliminates one or more unwanted organic compounds on said human hair.

19. The method according to claim 18, wherein said unwanted organic compound is sebum.

20. The method according to claim 15, wherein said one or more water-insoluble compounds are selected from the group consisting of V₂O₅, CeO₂, Nb₂O₅, WO₃, Na₄W₁₀O₃₂, MoO₃, MoS₂, RuO₂, Mn₂O₃, Bi₂O₃, Fe₂O₃, CuInS₂, CuIn₅S₈, Ag, Cu, Au, and Ni.

21. The method according to claim 15, wherein said one or more water-insoluble sulphides, carbides, and phosphides are selected from the group consisting of GaP, SiC, CdS and TiS.

22. The method according to claim 15, wherein said one or more water-insoluble composites are selected from the group consisting of TiO₂+ZnO, TiO₂+CuO, TiO₂+RuO₂, TiO₂+SnO₂, TiO₂+MoO₃, TiO₂+WO₃, TiO₂+GeO₂, TiO₂+MoO₃, TiO₂+WO₃, TiO₂+GeO₂, and TiO₂ surface-coated with one or more compounds comprising at least one element selected from the group consisting of Al, Zn, Zr, Cr, V, Nb, Fe, Cu, Co, Ni, and Mn.

23. The method according to claim 15, wherein the amount of said at least one photocatalyst is between 0.1 and 20% by weight relative to the total weight of said hair composition.

24. The method according to claim 15, wherein said hair composition comprises an aqueous or aqueous-alcoholic medium.

25. The method according to claim 15, wherein said hair composition further comprises one or more additives selected from the group consisting of a thickener, fragrance, pearlescent agent, preserving agent, sunscreen, anionic surfactant, nonionic surfactant, amphoteric surfactant, cationic surfactant, anionic polymer, nonionic polymer, amphoteric polymer, cationic polymer, protein, protein hydrolysate, ceramide, pseudoceramide, C₁₆-C₄₀ linear-chain fatty acid, C₁₆-C₄₀ branched-chain fatty acids hydroxy acid, vitamin, provitamin, silicone, plant oil, mineral oil, synthetic oil, and an anti-dandruff agent.

26. A cosmetic hair composition comprising, in a cosmetically acceptable medium, at least one photocatalyst selected from the group consisting of:
1) one or more water-insoluble compounds selected from the group consisting of MoS₂, RuO₂, Mn₂O₃, Bi₂O₃, CuInS₂, CuIn₅S₈, and a compound comprising at least one element selected from columns Vb, VIIb, VIII (2nd and 3rd columns starting from the left), and Va of the Periodic Table of Elements;
2) one or more water-insoluble sulphides and phosphides comprising at least one element selected from columns IIb, IIIa, IVa, and IVb of the Periodic Table of Elements; and
3) one or more water-insoluble composites selected from the group consisting of TiO₂+ZnO, TiO₂+CuO, TiO₂+RuO₂, TiO₂+SnO₂, TiO₂+MoO₃, TiO₂+WO₃, TiO₂+GeO₂, TiO₂+MoO₃, TiO₂+WO₃, TiO₂+GeO₂, and TiO₂ surface-coated with one or more compounds comprising at least one element selected from the group consisting of V, Nb, Cu, Mn.

27. The cosmetic hair composition according to claim 26, wherein said one or more water-insoluble compounds are selected from the group consisting of V₂O₅, Nb₂O₅, RuO₂, Mn₂O₃, Bi₂O₃, Co30₄, CuInS₂, and CuIn₅S₈.

28. The cosmetic hair composition according to claim 26, wherein said one or more water-insoluble sulphides and phosphides are selected from the group consisting of GaP, CdS, and TiS.

29. A method for decreasing, delaying, or decreasing and delaying regreasing of human hair, said method comprising:
applying said cosmetic hair composition as claimed in claim 26 to said human hair, wherein said human hair is dry human hair.

30. The method as claimed in claim 29, further comprising massaging said human hair with one or more fingers.

31. The method as claimed in claim 29, further comprising drying said human hair.

32. A method for decreasing, delaying, or decreasing and delaying regreasing of human hair, said method comprising:
applying a hair composition to said human hair, wherein said hair composition comprises at least one photocatalyst selected from the group consisting of:
1) one or more water-insoluble compounds comprising at least one element selected from columns IIb, IIIb, IVb, Vb, VIb, VIIb, VIII (3 columns), Ib, and Va of the Periodic Table of Elements;
2) one or more water-insoluble sulphides, carbides and phosphides comprising at least one element selected from columns IIb, IIIa, IVa, and IVb of the Periodic Table of Elements; and
3) one or more water-insoluble composites comprising at least one element selected from the group consisting of titanium, zinc, silicon, aluminium, magnesium, sodium, calcium and zirconium.

33. The method as claimed in claim 32, further comprising massaging said human hair with one or more fingers.

34. The method as claimed in claim 32, further comprising drying said human hair.
